(19) United States
(12) Patent Application Publication
(10) Pub. No.: US 2014/0176342 A1
(43) Pub. Date: Jun. 26, 2014

(54) Patient Environment with Accelerometer

(71) Applicants: Matthew Hopcroft, San Francisco, CA (US); Jerome Rolia, Kanata (CA); Sharad Singhal, Belmont, CA (US); Charles Edgar Bess, Frisco, TX (US); Henri J. Suermondt, Sunnyvale, CA (US)

(72) Inventors: Matthew Hopcroft, San Francisco, CA (US); Jerome Rolia, Kanata (CA); Sharad Singhal, Belmont, CA (US); Charles Edgar Bess, Frisco, TX (US); Henri J. Suermondt, Sunnyvale, CA (US)

(21) Appl. No.: 14/238,687

(22) PCT Filed: Sep. 29, 2011
(86) PCT No.: PCT/US2011/053999

§ 371 Date: Feb. 12, 2014

Related U.S. Application Data
(60) Provisional application No. 61/534,875, filed on Sep. 14, 2011.

(51) Int. Cl. A61B 5/00

(52) U.S. Cl. CPC A61B 5/0015
USPC 340/870.02

(57) ABSTRACT

A system includes a patient environment having a first surface and a second surface, an accelerometer mounted to the first surface and configured to capture physiological data of a patient in contact win the second surface, and a connection to transmit the physiological data to a processing system.

### BACKGROUND

The monitoring and diagnosing of many health conditions is generally accomplished with specialized equipment and trained operators, typically doctors and nurses, in a health care facility. Some health conditions Involve repeated visits to the health care facility, expensive equipment, and supplies or consumables (e.g., ECG leads). Other health conditions may be monitored in a patient's home but use equipment with wires and sensors that are directly attached to the patient. In either case, a patient may be unlikely to use these solutions on a continuous basis due to the inconvenience of visiting a health care facility and the intrusiveness of the home based equipment. Moreover, the intrusiveness of such equipment (whether in the home or a healthcare facility) may be substantial, thereby affecting patient satisfaction, rest, sleep, and potential recovery. Even if doctors are trained to diagnose internal conditions (e.g., by listening to stethoscope recordings of patients with known conditions), doctors are generally not available to monitor patients on a continuous basis.

### BRIEF DESCRIPTION OF THE DRAWINGS

FIGS. 1A-1C are schematic diagrams illustrating embodiments of patient environments with accelerometers for capturing physiological data of patients from the patent environments.

FIG. 2 is a schematic diagram illustrating one embodiment of capturing physiological data of a patient from a patent environment.

FIG. 3 is a flow chart Illustrating one embodiment of a method for capturing and providing physiological data with an accelerometer.

FIG. 4 is a signal diagram illustrating one embodiment of physiological data captured by an accelerometer.

FIG. 5 is a flow chart illustrating one embodiment of a method for processing physiological data from an accelerometer.

FIG. 6 is a block diagram illustrating one embodiment of a processing environment.

### DETAILED DESCRIPTION

In the following detailed description, reference is made to the accompanying drawings, which form a part hereof, and in which is shown by way of illustration specific embodiments in which the disclosed subject matter may be practiced. If is to be understood that other embodiments may be utilized and structural or logical changes may be made without departing from the scope of the present disclosure. The following detailed description, therefore, is not to be taken in a limiting sense, and the scope of the present disclosure is defined by the appended claims.

As described herein, a system includes a patient environment (e.g., a bed, a chair; or a wheelchair) instrumented with one or more highly sensitive accelerometers to monitor and detect various aspects of the health and condition of a patient. The accelerometers detect physiological data of a patient that transfers from the patient to the accelerometers through the patient environment (e.g. through the bed, chair, or wheelchair) rather than through a direct, connection from the patient to the accelerometer. The accelerometers provide physiological data of a patient to one or more processing systems that defect and/or infer specific features of the physiological data and the patients surroundings. The processing systems notify the patient, heath care providers, or other suitable persons of the presence of the features. As a result, the system provides non-intrusive, low cost, and highly available health care monitoring of patients in a home, an outpatient facility, or healthcare facility.

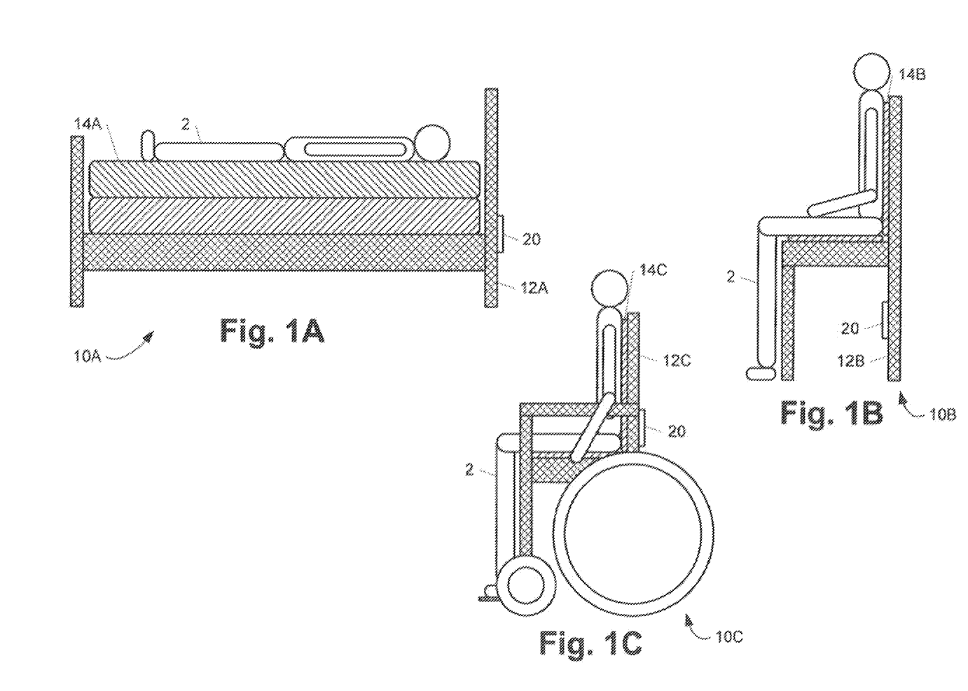

As used herein, the term patient environment includes a bed, chair, wheelchair, an examination table, or other suitable apparatus with one or more support surfaces configured for a patient to assume a relatively stationary position (e.g., lying and/or sitting). FIGS. 1A-1C are schematic diagrams illustrating embodiments 10A, 10B, and 10C of patent environments 10 with corresponding accelerometers 20 for capering physiological data of patients 2 from patent environments 10A, 10B, and 10C. During operation, accelerometers 20 capture physiological data from the vibrations generated by the internal and external body functions of patient 2 in contact with a patient environment 10. Accelerometers 20 also capture vibrations from the surroundings of the corresponding patient environment 10 (e.g., footsteps, door openings, etc.).

The human body can be described as a mechanical system with thousands of moving parts where every one of the parts can create a mechanical vibration from muscle movements or other body functions. These mechanical vibrations result from ongoing homeostatic processes such as breathing or beating of the heart These mechanical vibrations also result from additional muscle movement such as turning over in the bed or squirming in pain. Many of the vibrations of a body are extremely small and/or occur slowly.

As used herein, direct contact with regard to patient 2 refers to a physical connection of at least a portion of the body of patient 2 with a surface 14 of a patent environment 10. Patient 2 includes any clothing or other garments worn on the body, and surface 14 may include any bedding, cushions, upholstery, or other material that is exposed on surface 14. The direct contact of patient 2 may involve the clothing end/or skin of the patient 2 physically touching the bedding, cushions, upholstery, or other material on surface 14 to cause vibrations from Internal and external body motions of patent 2 to transfer from patient 2 to surface 14.

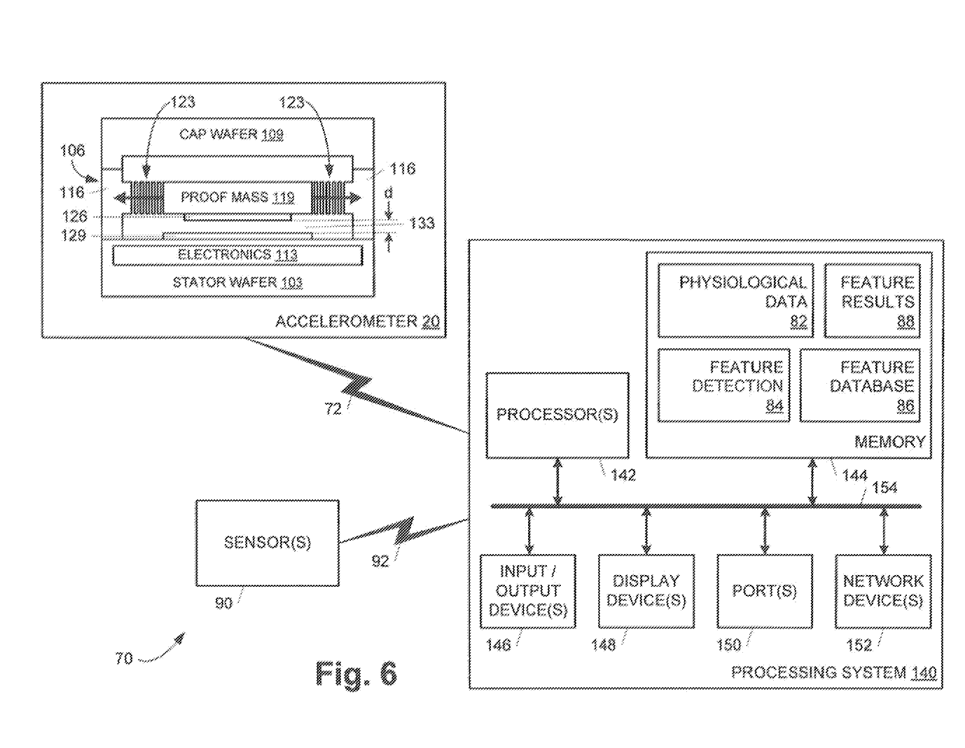

When in direct contact with a patient environment 10 (e.g., mounted on a patient environment 10), accelerometer 20 captures physiological data that represents the vibrations generated by patient 2 on patent environment 10 and transmitted to accelerometer 20 through the patient environment 10. As used herein, physiological data refers to a set of data values that collectively represent the frequency and amplitude of the vibrations defected by accelerometer 20 over time. From the physiological data, a processing system, such as processing system 140 shown in FIG. 6 and described in additional detail below, determines a range of health information including heart rate, respiration rate, and other specific features in the physiological data.

As used herein, direct contact with regard to accelerometer 20 refers to a physical connection of at least a portion of accelerometer 20 with a surface 12 of a patient environment 10. Accelerometer 20 includes any housing (not shown) that contains or supports accelerometer 20 any materials (not shown) used to mount or affix accelerometer 20 to surface 12. Accordingly, the direct contact of accelerometer 20 may involve the housing or materials used to mount or affix accelerometer 20 physically touching surface 12 to cause vibrations from internal and external body motions of patient 2 to transfer from surface 14 to accelerometer 20. Accelerometer 20 does not directly connect to patient 2 in contrast with other types of sensors that may use ECG leads, a stethoscope, a pulse oximeter, or a blood pressure cuff.

Accelerometer 20 includes ultra-high sensitivity microfabricated accelerometer technology with three-phase sensing as described by U.S. Pat. Nos. 8,882,019, 7,142,500, and 7,484,411 and incorporated by reference herein in their entirety. Accelerometer 20 is a sensor which detects acceleration, i.e., a change in a rate of motion, with a high sensitivity and dynamic range. Because of the three-phase sensing technology, accelerometer 20 may sense acceleration levels as low as 10's of nano-gravities and may be manufactured and housed in a device that has typical dimensions of 5×5×0.5 mm or less using Micro-Electro-Mechanical-Systems (MEMS) technology. The combination of high sensitivity and small device size enabled by three-phase sensing techniques allows accelerometer 20 to unobtrusively capture physiological data transmitted from a patient 2 through a patient environment 10 without direct contact between any portion of accelerometer 20 and patient 2. For example, no leads are used to connect accelerometer 20 to patient 2. In addition, the sensitivity of accelerometer 20 allows physiological data to be captured such that specific features of physiological conditions of patient 2 can be defected by a processing system. These features include not only cardiac pulse and respiratory rate but specific conditions such as arrhythmia. The features may indicate whether the patient is restlessness, present in a patient environment 10 (e.g., in bed), about to fall, needs to go to the bathroom, in pain, or being disturbed by others in the room. Additional details of accelerometer 20 are shown and described with reference to FIG. 6 below.

In FIG. 1A, bed 10A includes a frame, box spring, a mattress, and bedding (not shown) that transmit vibrations of patient 2 from a surface 14A in direct contact with patient 2 (i.e., the top surface of the mattress) to a surface 12A of the bed frame on which accelerometer 20 is directly connected. Accelerometer 20 captures the physiological date from the vibrations transmitted from surface 14A through the mattress, box spring, and bed frame (i.e., a plurality of materials) to surface 12A. Patient 2 is not in direct contact with surface 12A, and no portion of accelerometer 20 is in direct contact with patient 2—i.e., accelerometer 20 does not include any wired or wireless connections to patient 2 other than through bed 10A itself.

In FIG. 18, chair 10B includes a chair frame and cushions or upholstery that transmit vibrations of patient 2 from a surface 148B in direct contact with patient 2 (i.e., the top surface of the cushions or upholstery) to a surface 12B of the chair frame on which accelerometer 20 is directly connected. Accelerometer 20 captures the physiological data from the vibrations transmitted from surface 14B through the cushions or upholstery and chair frame (i.e., a plurality of materials) to surface 12B. Patient 2 is not in direct contact with surface 12B, and no portion of accelerometer 20 is in direct contact with patient 2—i.e., accelerometer 20 does not include any wired or wireless connections to patient 2 other than through chair 10B itself.

Similarly in FIG. 1C, wheelchair 10C includes a wheelchair frame and cushions or upholstery that transmit vibrations of patient 2 from a surface 14C in direct contact with patient 2 (i.e., the top surface of the cushions or upholstery) to a surface 12C of the wheelchair frame on which accelerometer 20 is directly connected. Accelerometer 20 captures the physiological data from the vibrations transmitted torn surface 14C through the cushions or upholstery and wheelchair frame (i.e., a plurality of materials) to surface 12C. Patient 2 is not in direct contact with surface 12C, and no portion of accelerometer 20 is in direct contact with patient 2—i.e., accelerometer 20 does not include any wired or wireless connections to patient 2 other than through wheelchair 10C itself.

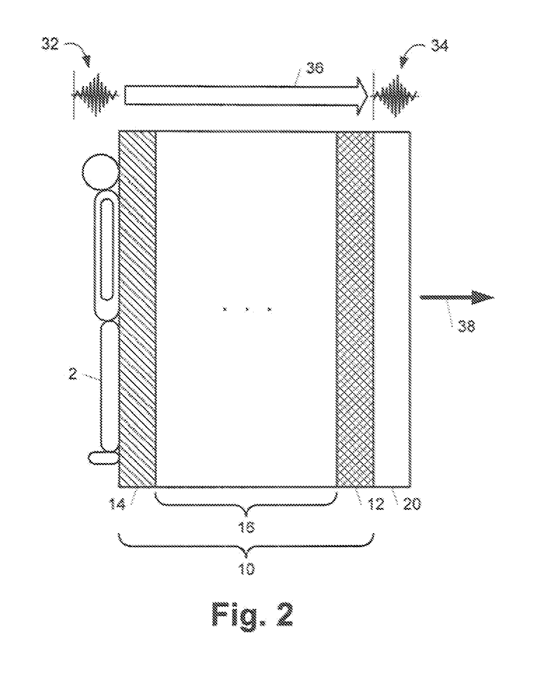

FIG. 2 is a schematic diagram illustrating one embodiment of capturing physiological data of patient 2 from patent environment 10. FIG. 2 illustrates the transmission of vibrations 32 of patient 2 that are captured by accelerometer 20 as physiological data 34 as indicated by an arrow 36. In particular, patient 2 generates vibrations 32 which are transferred to a surface 14 in direct contact with patient 2. Surface 14 transfers vibrations 32 though a plurality of materials 16 of patient environment 10 to surface 12. Plurality of materials 16 is disposed between surface 14 and surface 12 and includes any suitable type and combination of materials for patient environment 10 as set forth by embodiments 10A, 10B, and 10C described above. Surface 12 transfers vibrations 32 to accelerometer 20 where vibrations 32 are captured as physiological data 34 and provided to a processing system over any suitable wired or wireless connection 38.

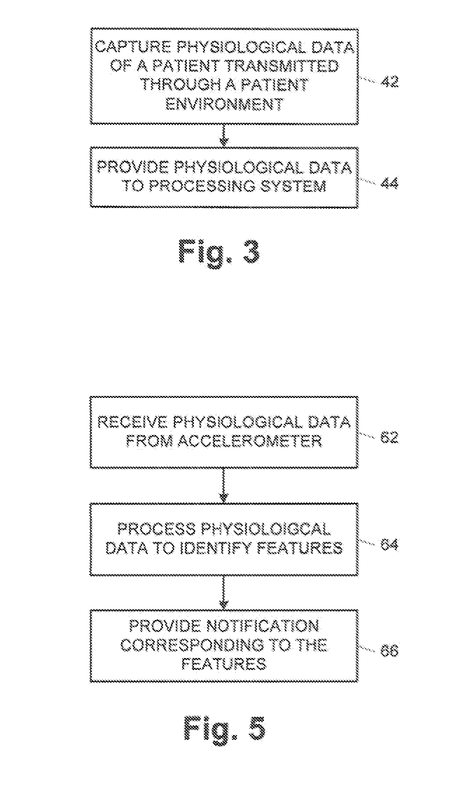

The functions of accelerometer 20 are further illustrated in FIG. 3 which is a flow chart illustrating one embodiment of a method for capturing and providing physiological data with accelerometer 20. In FIG. 3, accelerometer 20 captures physiological data of a patient 2 that is transmitted through a patient environment 10 as indicated in a block 42. The physiological data may be transmitted from patient 2 through a plurality of materials of the patient environment 10 before reaching accelerometer 20 as described above.

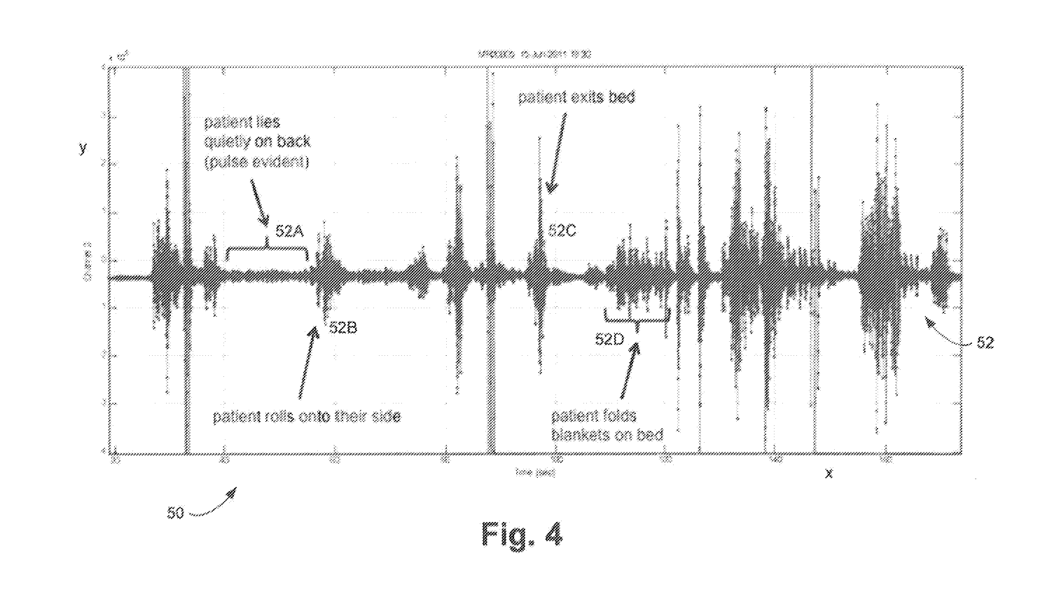

FIG. 4 is a signal diagram 50 illustrating one embodiment of physiological data 52 of patient 2 captured by accelerometer 20 while patient 2 is lying in a bed 10A. The amplitude of the vibrations of physiological data 52 is plotted in the y axis over time in the x axis. Various features of the health condition of patient 2 appear in physiological data 52. For example, the pulse rate is apparent in a portion 52A of data 52 and actions of patient 2 such as rolling over, exiting bed 10A, and folding blankets on bed 10A are apparent in portions 52B, 52C, and 52D of data 52, respectively. Other information about patient 2 and any events and actions occurring in and around bed 10A are evident in physiological data 52.

The typical level of the vibrations detected by accelerometer 20 in this example is a few μf (i.e., 1×10⁻⁶ g). Accordingly, physiological data 52 includes features present only in data captured by a high sensitivity accelerometer 20. Physiological data 52 data can be analysed by a processing system using time and/or frequency domain information and correlated by the processing system to specific health conditions of patient 2.

Referring back to FIG. 3, accelerometer 20 provides the physiological data to a processing system as indicated in a block 44. Accelerometor 20 may provide physiological data to the processing system by continuously transferring the data to the processing system or by storing the data in computer readable medium (not shown) for periodic transmittal or retrieval by the processing system. Accelerometer 20 may provide the physiological data to a processing system using any suitable type of wired and/or wireless connections, such as connection 72 shown in FIG. 6 and described in additional detail below.

The functions of one or more processing systems (e.g., processing system 140 shown in FIG. 6) are illustrated in FIG. 5 which is a flow chart illustrating one embodiment of a method for processing physiological data from accelerometer 20. The method of FIG. 5 will be described with reference to processing system 140 shown in FIG. 8.

In FIG. 5, processing system 140 receives physiological data from accelerometer 20 as Indicated in a block 62. Processing system 140 may receive the data as a continuous or periodic stream from accelerometer 20 or may retrieve the data by accessing the data from a computer readable medium (not shown) of the accelerometer 20. Processing system 140 processes the physiological data to identify features of a condition of patient 2 in physiological data as indicated in a block 64. Processing system 140 may use frequency domain or time domain analysis to identify the features. Processing system 140 may also detect features by correlating known patterns from a feature database (e.g., feature database 86 shown in FIG. 6) with features in the physiological data. Processing system 140 may also detect features using both the physiological data and sensor data from other sensors (e.g., sensors 90 shown in FIG. 6) in direct or indirect contact with patient 2.

Examples of features detectable by processing system 140 using the physiological data will now be described. In a first example, accelerometer 20 on bed 10A measures the activity of patient 2 while the patient sleeps. Processing system 140 identifies features that represent the quality of the sleep of patient 2 by examining threshold levels of the physiological date. The features may include apnea (i.e., snoring) or involuntary muscle movement (e.g., restless leg syndrome). Processing system 140 records sleep interruptions and may correlate the interruptions to specific external sources such as a person entering the room or adjusting bed 10A. Processing system 140 may also discern subtle variations in activity levels of patient 2 that correspond to discomfort or pain of patient 2. In particular, processing system 140 may determine the effectiveness of pain medication or other relief on patient 2 based on the level of patient movement during sleep and the amount of time the patient is asleep, awake and out of bed.

In another example, processing system 140 identifies features of the cardiac rhythms of patient 2. These features include pulse rate as well as arrhythmia and other heart conditions. Processing system 140 performs time and frequency domain analysis on the physiological data and compares the analyzed data to a database of cardiac conditions (e.g., feature database 86 shown in FIG. 6) to identify the features in some embodiments.

In a further example, the features identified by processing system 140 include muscle tremors of patient 2. The muscle tremors detected in the physiological data may reflect a normal level of muscle tremors in patient 2 or an increase of muscle tremors over time that may be indicative of certain neurological conditions. Processing system 140 measures and quantifies the small changes in muscle tremor activity in this example.

Other examples of features detectable by processing system 140 using the physiological data include lack of activity indicating that the patient has left the bed, prolonged lack of turning over (which may put the patient at risk for pressure ulcers), restlessness, and movement patterns characteristic of a specific stage of sleep.

Processing system 140 provides a notification corresponding to the identified features as indicated in a block 66. Processing system 140 may provide the notification to patient 2 or any suitable interested person associated with patient 2 such as healthcare professionals or family, friends, or others having a relationship with patient 2. Processing system 140 may provide notifications at any suitable time and in any suitable way. For example, processing system 140 may provide art immediate notification by displaying the feature, an identification of the feature, or a notice that a feature of interest has been defected to an Interested person, Processing system 140 may also store a log or other report of identified features (e.g., feature results 88 shown in FIG. 6) for later retrieval by an interest person. The log or other report may include comparisons with other patients with similar demographics whose physiological data has also been captured using an accelerometer 20.

The notifications of processing system 140 may include conclusions reached eased the specific features defected by processing system 140. For example, a notification could note that a patient is in bed, about to fall, needs to go to the bathroom, in pain, or being disturbed by others in the room.

Processing system 140 may be configured according to a set of reporting policies that determine how notifications of identified features are disseminated. For example, notifications may be generated only when the identified features meet some statistically determined threshold with respect to the physiological data of patient 2 or the physiological data of other patients captured using another accelerometer 20. The threshold may be set differently for different interested persons (e.g., a family member may receive a notification at a lower threshold than a doctor).

FIG. 6 is a block diagram illustrating one embodiment of a processing environment 70. Processing environment 70 includes accelerometer 20 in communication with processing system 140 across a connection 72. One or more additional sensors 80 in direct contact with a patient 2 may also be in communication with processing system 140 across one or more connections 92.

In the embodiment of FIG. 6, accelerometer 20 includes three layers, or “wafers.” In particular, accelerometer 20 includes a stator wafer 103, a rotor wafer 106, and a cap wafer 109. Stator wafer 103 includes electronics 113 that may be electrically coupled to various electrical components in rotor wafer 106 and cap wafer 109. Also, electronics 113 may provide output ports for coupling to electronic components external to accelerometer 20.

Rotor wafer 106 includes support 116 that is mechanically coupled to a proof mass 119. Although the cross-sectional view of accelerometer 20 is shown, according to one embodiment, support 116 as a portion of rotor wafer 106 surrounds proof mass 119. Consequently, in one embodiment stator wafer 103, support 110, and cap wafer 109 form a pocket within which proof mass 119 is suspended.

Together, stator wafer 103, support 116, and cap wafer 109 provide a support structure to which proof mass 119 is attached via a compliant coupling. The compliant coupling may, in one embodiment, comprise high aspect ratio flexural suspension elements 123 described In U.S. Pat. No. 6,882,019.

Accelerometer 20 further includes a first electrode array 128 that is disposed on proof mass 119. In one embodiment, first electrode array 126 is located on a surface of proof mass 110 that is opposite the upper surface of stator wafer 103. The surface of the proof mass 110 upon which the first electrode array 126 Is disposed is a substantially flat surface.

A second electrode array 129 is disposed on a surface of stator wafer 103 facing opposite first electrode array 128 disposed on proof mass 119, Because proof mass 126 is suspended over stator wafer 103, a substantially uniform gap 133 (denoted by d) is formed between first electrode array 126 and second electrode array 129. The distance d may comprise, for example, anywhere from 1 to 3 micrometers, or it may be another suitable distance.

Proof mass 119 is suspended above stator wafer 103 so that first electrode array 126 and second electrode array 129 substantially fall into planes that are parallel to each other and gap 133 is substantially uniform throughout the overlap between first and second electrode arrays 126 and 129. In other embodiments, electrode arrays 126 and 129 may be placed on other surfaces or structures of stator wafer 103 or proof mass 119.

High aspect ratio flexural suspension elements 123 offer a degree of compliance that: allows proof mass 119 to move relative to the support structure of accelerometer 20 (not shown). Due to the design of flexural suspension elements 123, the displacement of proof mass 119 from a rest position is substantially restricted to a direction that is substantially parallel to second electrode array 129, which is disposed on the upper surface of stator wafer 103. Flexural suspension elements 123 are configured to allow for a predefined amount of movement of proof mass 119 in a direction parallel to second electrode array 129 such that gap 133 remains substantially uniform throughout the entire motion to the extent possible. The design of flexural suspension elements 123 provides for a minimum amount of motion of proof mass 119 in a direction orthogonal to second electrode array 129 while allowing a desired amount of motion in the direction parallel to second electrode array 129.

As proof mass 119 moves, capacitances between first and second electrode arrays 126 and 129 vary with the shitting of the arrays with respect to each other. Electronics 113 and/or external electronics are employed to detect or sense the degree of the change in the capacitances between electrode arrays 128 and 129. Based upon the change in the capacitances, such circuitry can generate appropriate signals that are proportional to the vibrations from patient 2 experienced by accelerometer 20.

The operation of accelerometer 20 is enhanced by the use of three-phase sensing and actuation as described by U.S. Pat. Nos. 6,882,019 and 7,484,411. Three-phase sensing uses an arrangement of sensing electrodes 128 and 129 and sensing electronics 113 to enhance the output signal of accelerometer 20 and allow for the sensitivity to be maximized in a desired range. It also allows the output of accelerometer 20 to be “reset” to zero electronically when the sensor is in any arbitrary orientation.

Processing system 140 represents any suitable processing device, or portion of a processing device, configured to implement the functions of the method shown in FIG. 3 and described above. A processing device may be a laptop computer, a tablet computer, a desktop computer, a server, or another suitable type of computer system. A processing device may also be a mobile telephone with processing capabilities (i.e., a smart phone) or another suitable type of electronic device with processing capabilities. Processing capabilities refer to the ability of a device to execute instructions stored in a memory 144 with at least one processor 142. Processing system 140 represents one of a plurality of processing systems in a cloud computing environment in one embodiment.

Processing system 140 includes at least one processor 142 configured to execute machine readable instructions stored in a memory system 144. Processing system 140 may execute a basic input output system (BIOS), firmware, an operating system, a runtime execution environment, and/or other services and/or applications stored in memory 144 (not shown) that includes machine readable instructions that are executable by processors 142 to manage the components of processing, system 140 and provide a set of functions that allow other programs to access and use the components. Processing system 140 stores physiological data 82 received from accelerometer 20 in memory system 144 along with a feature detection unit 84 that performs the method of FIG. 5 described above. Processing system 140 further stores a feature database 86 and feature results 88 in some embodiments.

Processing system 140 may also include any suitable number of input/output devices 14b, display devices 148, ports 150, and/or network devices 152. Processors 142, memory system 144, input/output devices 148, display devices 148, ports 150, and network devices 152 communicate using a set of interconnections 154 that includes any suitable type, number, and/or configuration of controllers, buses, interfaces, and/or other wired or wireless connections. Components of processing system 140 (for example, processors 142, memory system 144, input/output devices 148, display devices 148, ports 150, network devices 152, and interconnections 154) may be contained in a common housing with accelerometer 20 (not shown) or in any suitable number of separate housings separate from accelerometer 20 (not shown).

Each processor 142 is configured to access and execute instructions stored in memory system 144 including feature detection unit 84. Each processor 142 may execute the instructions in conjunction with or in response to information received from input/output devices 146, display devices 148, ports 150, and/or network devices 152. Each processor 142 is also configured to access and store data, including physiological data 82, feature database 86. and feature results 88, in memory system 144.

Memory system 144 includes any suitable type, number, and configuration of volatile or non-volatile storage devices configured to store instructions and data. The storage devices of memory system 144 represent computer readable storage media that store computer-readable and computer-executable instructions including feature detection unit 84. Memory system 144 stores instructions and data received from processors 142, input/output devices 146, display devices 148, ports 150, and network devices 152. Memory system 144 provides stored instructions and data to processors 142, input/output devices 148, display devices 148, ports 150, and network devices 152. Examples of storage devices in memory system 144 include hard disk drives, random access memory (RAM), read only memory (ROM), flash memory drives and cards, and other suitable types of magnetic and/or optical disks.

Input/output devices 146 include any suitable type, number, and configuration of input/output devices configured to input instructions and/or data from a user to processing system 140 and output instructions and/or data from processing system 140 to the user. Examples of input/output devices 146 include a touchscreen, buttons, dials, knobs, switches, a keyboard, a mouse, and a touchpad.

Display devices 148 include any suitable type, number, and configuration of display devices configured to output image, textual, and/or graphical information to a user of processing system 140. Examples of display devices 148 include a display screen, a monitor, and a projector. Ports 150 include suitable type, number, and configuration of ports configured to input instructions and/or data from another device (not shown) to processing system 140 and output instructions and/or data from processing system 140 to another device.

Network devices 152 include any suitable type, number, and/or configuration of network devices configured to allow processing system 140 to communicate across one or more wired or wireless networks (not shown). Network devices 152 may operate according to any suitable networking protocol and/or configuration to allow Information to be transmitted by processing system 140 to a network or received by processing system 152 from a network.

Connection 72 includes any suitable type and combination of wired and/or wireless connections that allow accelerometer 20 to provide physiological data 82 to processing system 140. Connection 72 may connect to one or more ports and/or one or more network devices 152 of processing system 140. For example, connection 72 may comprise a wireless network connection that includes a wireless network, device (not shown) that transmits physiological data 82 from accelerometer 20 to processing system 140. As another example, connection 72 may comprise a cable connected from accelerometer 20 to a port 150 to transmit physiological data 82 from accelerometer 20 to processing system 140.

Sensors 90, if present, may provide sensor data of patient 2 (not shown) to processing system 140 in addition to physiological data 82. Sensors 90 may be placed in direct or indirect contact with patient 2 to generate the sensor data. Processing system 140 may use the sensor data In conjunction with physiological data 82 to defect and/or confirm specific features of a patient condition.

Referring back to FIGS. 1A-1C and 2, an optimal placement of accelerometers 20 may be determined using various methods to allow increase the detectability of physiological data 82 by accelerometers 20. In at least some of the methods, accelerometers 20 are initially affixed at one location in a patient environment 10 and then signals are observed at accelerometers 20. Accelerometers 20 are then affixed at one or more different locations and the signals observed again at accelerometers 20. An adjustable clamp (not shown) may be used to temporarily affix accelerometers 20.

In one method, the natural resonance of patient environment 10 is measured by accelerometers 20 without any simulated external stimulation. Patient environment 10, including the materials therein, may act to amplify or attenuate different, aspects of signals. The locations of accelerometer 20 that provide the best signet reception or the least disruption at frequencies of interest may be selected as the optimal locations.

In another method, a vibration simulator is employed to provide known stimulus to patient environment 10. The stimulus may represent a particular waveform such as a sine wave. Such a wave can be used to find a location for accelerometers 20 that causes the least interference with the signal. Alternatively, the simulator may mimic waveforms that correspond to specific patient features to be identified from physiological data. The locations of accelerometer 20 that cause the least interference with the signal or that lead to the best quality of specific patient features may be selected as the optimal locations.

When repeatedly moving the locations of accelerometers 20, care may be taken to place accelerometers 20 at discrete intervals that correspond to the dimension of accelerometers 20. Joints and midpoints between joints of patient environment 10 may be considered. By doing so, all possible resonance behaviors may be observed. Other sensors 90, e.g., light sensors or microphones, may also have locations chosen to provide protection from locations that may incur unintended impacts or that may otherwise limit their effectiveness.

Accelerometers 20 and processing system 140 may also be used to capture of additional information for calibrating or training feature detection unit 84 according to several techniques. In one method, the correlation of standard patient medical records (electronic and paper) for a patient 2 or group of patients 2 with readings of accelerometers 20 is performed. These records include information about patient demographics and readings using other types of sensors. Next, trained observers, such as nurses or medical students, may record detailed observations of the patient 2 or group of patients 2, such as whether a patient 2 gets Into or out of a bed 10A. Further, patients 2 may be surveyed directly and asked to record their own observations, in addition, video footage, measurements of sound, or other measurements may also be recorded and compared with readings of accelerometers 20.

In another method, patients 2 in patient environments 10 may be asked to mimic a series of actions that include physiological conditions and events of interest, in some cases, the observations may be recorded in real time using one or more input devices 148. Information may be input directly to system 70 or to some other network accessible by system 70. The information may be depersonalized and methods may be offered for patients to specify how the information may be used.

The above embodiments may advantageously enable non-intrusive, continuous, long-term, inexpensive measurements of physiological functions which may otherwise be difficult or time consuming for health care personnel to make. The embodiments may also provide the ability to communicate results to Interested persons based on configurable policies. This may allow the above embodiments to participate in health care processes for patients in an active and timely manner.

Although specific embodiments have been illustrated and described herein for purposes of description of the embodiments. It will be appreciated by those of ordinary skill in the art that a wide variety of alternate and/or equivalent implementations may be substituted for the specific embodiments shown and described without departing from the scope of the present disclosure. Those with skill in the art will readily appreciate that the present disclosure may be Implemented In a very wide variety of embodiments. This application is intended to cover any adaptations or variations of the disclosed embodiments discussed herein. Therefore, it is manifestly intended that the scope of the present disclosure be limited by the claims and the equivalents thereof.

What is claimed is:

1. A system comprising:
a patient environment having a first surface and a second surface;
an accelerometer mounted to the first surface, the accelerometer to capture physiological data of a patient in contact with the second surface; and
a connection to transmit the physiological data to a processing system.

2. The system of claim 1 wherein the patient is not in contact with the first surface while the accelerometer captures the physiological data.

3. The system of claim 1 wherein the patient environment is to transmit the physiological data between the first and the second surfaces.

4. The system of claim 1 wherein the patient environment includes a plurality of materials between the first surface and the second surface.

5. The system of claim 4 wherein the physiological data represents internal and external body movements of the patient transmitted through the plurality of materials.

6. The system of claim 1 wherein the patient environment includes one of a bed, a chain a wheelchair, or an examination table.

7. The system of claim 1 wherein the accelerometer includes a proof mass with a first electrode array suspended above a second electrode array disposed on a wafer.

8. A method performed by a processing system, the method comprising:
receiving at the processing system, physiological date of a patent in a patient environment from an accelerometer in direct contact with the patient environment but not in direct contact with the patient;
processing, at the processing system, the physiological data to identity a feature of a physiological condition of the patient; and
providing, at the processing system, a notification corresponding to the feature.

9. The method of claim 8 former comprising:
detecting, at the processing system, a correlation between the feature and sensor data captured by a sensor in direct contact with the patient.

10. The method of claim 8 further comprising:
providing the notification to at least one of the patient or a healthcare professional,

11. The method of claim 8 wherein the accelerometer includes three-phase sensing and actuation.

12. A system comprising:
a patient environment having a first surface to support a patient, a second surface, and a plurality of materials between the first surface and the second surface;
an accelerometer in contact with the second surface to capture physiological data of the patient transmitted through the first surface, the plurality of materials, and the second surface; and
a processing system to identify a feature of a physiological condition of the patient using the physiological data.

13. The system claim 12 wherein the accelerometer captures the physiological data without direct contact between any portion of the accelerometer and the patient.

14. The system claim 12 wherein the processing system is to perform one of frequency domain or time domain analysis to identity the feature.

15. The system claim 12 wherein the accelerometer detects changes in capacitances between a first electrode arrays disposed on a proof mass and a second electrode array disposed on a wafer.
